(19) United States
(12) Patent Application Publication
(10) Pub. No.: US 2024/0383881 A1
(43) Pub. Date: Nov. 21, 2024

(54) SYNTHETIC COMPOUND OF MYRICETIN AND NOBILETIN, AND PREPARATION METHOD THEREFOR

(71) Applicant: The Second People's Hospital of Shenzhen (Shenzhen Institute of Geriatrics), Shenzhen (CN)

(72) Inventors: Zhengzhi Wu, Shenzhen (CN); Liuyang Pu, Shenzhen (CN); Zhiyue Li, Shenzhen (CN); Yan Li, Shenzhen (CN); Zhanyan Liu, Shenzhen (CN); Guanbao Zhu, Shenzhen (CN); Limin Li, Shenzhen (CN); Yaqian Liu, Shenzhen (CN)

(21) Appl. No.: 18/403,555

(22) Filed: Jan. 3, 2024

(30) Foreign Application Priority Data
May 19, 2023 (CN) 2023105679713

(51) Int. Cl. C07D 407/12; A61P 7/00

(52) U.S. Cl. CPC C07D 407/12; A61P 7/00

(57) ABSTRACT

A synthetic compound of myricetin and nobiletin, and a preparation method therefor are provided, two clinically significant lead compounds of the myricetin and the nobiletin are modified, a new synthetic compound of myricetin and nobiletin is obtained. Compared with traditional western medicine, the new synthetic compound has significant advantages in treating comorbidities and has fewer adverse reactions. The reduction of serum uric acid and creatinine levels by the synthetic compound of myricetin and nobiletin is significantly higher than that of monomers myricetin and monomers nobiletin, which indicates that the synthetic compound of myricetin and nobiletin has a better uric acid lowering effect than monomeric medicines and has a significant improvement effect on hyperuricemia (HUA).

### TECHNICAL FIELD

The disclosure relates to the technical field of medicines, and particularly to a synthetic compound of myricetin and nobiletin (MNH), and a preparation method therefor.

### BACKGROUND

Hyperuricemia (HUA) is a major risk factor for the occurrence and development of gout, and an independent risk factor for a variety of cardiovascular and metabolic diseases, including diabetes, hypertension, atherosclerosis, and chronic kidney disease (CKD). Both medicine development and evaluation, as well as exploration of hyperuricemia and related disease mechanisms are inseparable from the establishment of experimental models of hyperuricemia. Given the convenience of feeding and obtaining experimental animals, rodents are currently the main animals used for studying hyperuricemia. In vivo, the metabolic process of uric acid is roughly as follows: adenine ribonucleotides, adenosine, inosine, hypoxanthine, xanthine, uric acid, sequentially. The increasing of the uric acid or its precursor substances can cause the increase of blood uric acid levels in vivo, and can also reduce the path of uric acid by inhibiting the further degradation of uric acid and the excretion of uric acid, so as to achieve the purpose of increasing blood uric acid. In this study, the principle of yeast extract-induced hyperuricemia model is adopted. In this situation, yeast extract contains rich proteins, nucleotides, and B vitamins, which can be fully hydrolyzed in vivo to produce nitrogenous bases (including purine bases and pyrimidine bases) and phosphoric acids. High doses of yeast in vivo can interfere with normal purine metabolism of the body and cause purine metabolism disorder, mainly manifested by increased xanthine oxidase activity to accelerate uric acid production.

Currently, western medicines for the treatment of hyperuricemia mainly include medicines that inhibit uric acid production such as febuxostat and allopurinol, new uric acid decomposition medicines such as rasburicase and pegloticase, and uric acid excretion medicines such as benzbromarone and lesinurad. Although the western medicine has a significant uric acid lowering effect, its efficacy in treating complications of the hyperuricemia is poor, and even adverse reactions show a certain degree of nephrotoxicity. In recent years, multiple research findings and clinical observations have shown that traditional Chinese medicine and ethnomedicine have significant therapeutic advantages in preventing and treating hyperuricemia and its complications, especially in alleviating complications. Myricetin is a common plant-derived flavonoid with a wide range of biological activities including strong antioxidant, anticancer, antidiabetic and anti-inflammatory effects. Previous clinical studies have reported that myricetin can significantly reduce serum uric acid (UA) levels in patients with hyperuricemia (Wang Yong et al., “Effects and significance of myricetin and puerarin (C₂₁H₂₀O₉) on hyperuricemia”, Nei Mongol Journal of Traditional Chinese Medicine, 2010, pages 9-10). Nobiletin, a citrus flavonoid isolated from citrus peel, has anti-inflammatory and anti-tumor activities. The half-maximal inhibitory concentration (IC₅₀) of nobiletin on organic anion transporter 4 (OAT4) of overexpressed monoclonal cell line is 0.556 micromoles per liter (μmol/L), which indicated that nobiletin has strong inhibitory effect on OAT4 (Wang Ze et al., “Effects of Chinese herb ingredients with different properties on OAT4, URAT1 and serum uric acid level in acute hyperuricemia mice”, Chinese Traditional and Herbal Drugs, 2019, pages 1157-1163). Given that the myricetin and the nobiletin have good uric acid lowering effects, it is of great theoretical significance and practical value to use the two types of compounds as lead compounds for structural modification and transformation to develop new uric acid lowering medicines.

### SUMMARY

The disclosure aims to provide a synthetic compound of myricetin and nobiletin, a preparation method therefor and a medical application thereof, so as to fully utilize the medicinal value of the synthetic compound in clinical practice. Through scientific experiments, it is found that the hyperuricemia (HUA) model of mice fed with yeast extract feed is successfully replicated. In this situation, the serum uric acid levels, creatinine, urea nitrogen levels, the kidney index and liver index of mice in the model group are significantly increased, and renal lesions are obvious. The synthetic compound can significantly reduce the levels of uric acid, creatinine, and urea nitrogen in hyperuricemia model mice, lower liver and kidney indexes, and improve renal pathological changes. In addition, the reduction levels of serum uric acid and creatinine of the synthetic compound are significantly higher than that of monomer myricetin and monomer nobiletin, which indicates that the synthetic compound has a better uric acid lowering effect than monomeric medicines and has a significant improvement effect on hyperuricemia. It provides a theoretical basis for the clinical development of the synthetic compound as uric acid lowering medicines.

A synthetic compound of myricetin (C₁₅H₁₀O₈) and nobiletin (C₂₁H₂₂O₈) is provided, and a structural general formula is shown in a formula E expressed as follows:

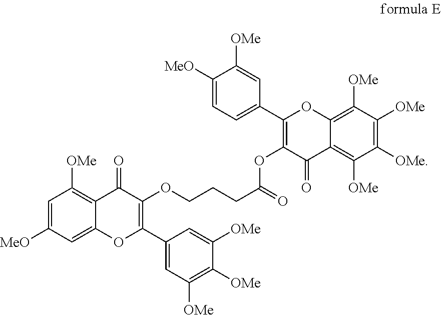

A preparation method for the synthetic compound of myricetin and nobiletin includes the following steps.

(1) mixing myricitrin (C₂₁H₂₀O₁₂) and potassium carbonate (K₂CO₃) in an acetonitrile (C₂H₃N) solution to obtain a first mixed solution, then adding dimethyl sulfate (Me₂SO₄) into the first mixed solution to perform a temperature control reaction to obtain a compound A, and dissolving the compound A in ethanol, followed by adding a concentrated hydrochloric acid (conc. HCl) to react to obtain a compound B. A chemical equation I is as follows:

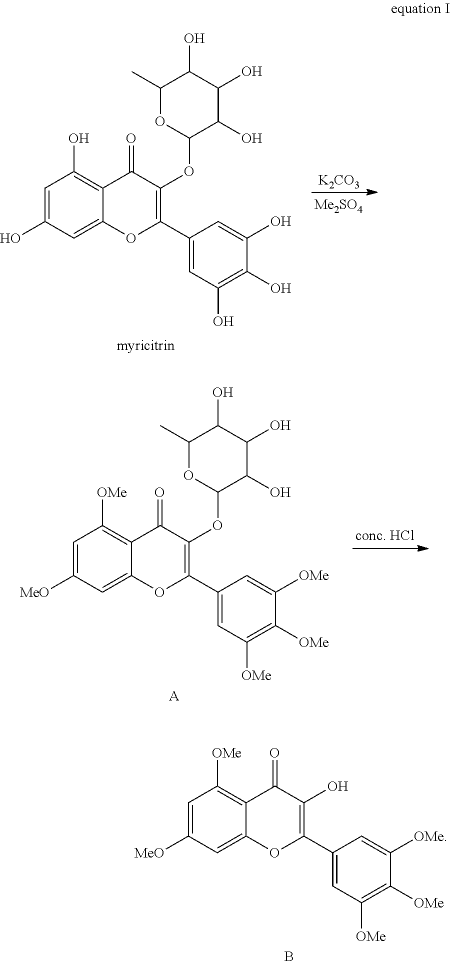

In the step (1), a molar ratio of the myricitrin to the potassium carbonate is in a range of 1:6-8, a volume mass ratio of the acetonitrile solution to the myricitrin is 50:1, and a molar ratio of the dimethyl sulfate to the potassium carbonate is 1:1. A volume ratio of the acetonitrile solution to the ethanol used to dissolve the compound A is 1:1, a mass concentration of the concentrated hydrochloric acid is 37% in terms of hydrogen chloride (HCl), and a molar ratio of the concentrated hydrochloric acid to the myricetin is 10:1.

(2) adding the potassium carbonate and BrCH₂CH₂CH₂CO₂Et into a N,N-dimethylformamide (C₃H₇NO) solution of the compound B to perform a temperature control reaction to obtain a compound C. A chemical equation II is as follows:

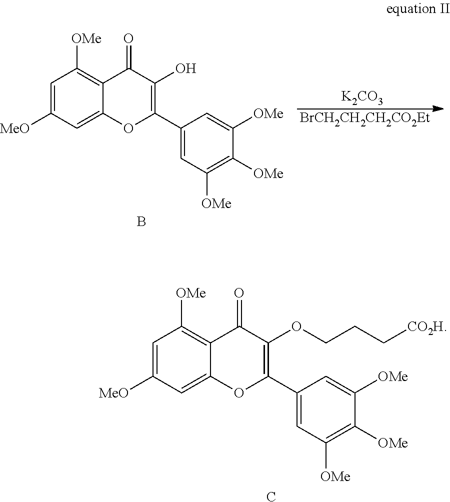

In the step (2), a volume mass ratio of the compound B to the N,N-dimethylformamide solution is in a range of 1:15-20, a molar ratio of the potassium carbonate to the compound B is 3:1, a molar ratio of the BrCH₂CH₂CH₂CO₂Et to the compound B is 2:1. A mass concentration of the aqueous solution of sodium hydroxide is in a range of 5%-40%, a molar ratio of the sodium hydroxide to the compound B is 2:1, and a molar concentration of the dilute hydrochloric acid is 4 moles per liter (mol/L).

(3) adding an aqueous solution of sodium carbonate (Na₂CO₃) and an aqueous solution of sodium carbonate of sodium bicarbonate (NaHCO₃) into a dichloromethane (CH₂Cl₂) and acetone (C₃H₆O) solution of the nobiletin to obtain a second mixed solution, followed by adding an aqueous solution of potassium peroxymonosulfate (HKO₆S) into the second mixed solution to obtain a compound D. A chemical equation III is as follows:

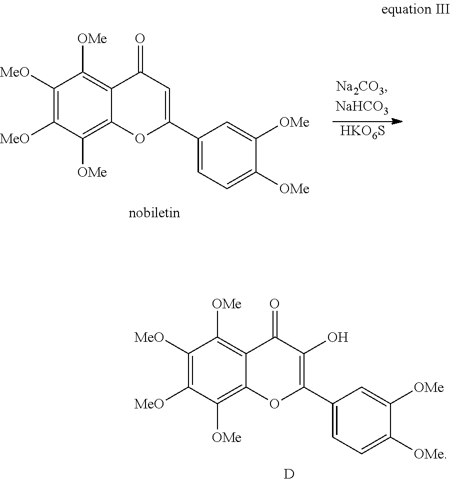

In the step (3), a volume mass ratio of the dichloromethane and acetone solution to the nobiletin is 175:1, a volume ratio of the dichloromethane to the acetone is 4:3, a mass concentration of the aqueous solution of sodium carbonate is 5%, a molar ratio of the sodium carbonate to the nobiletin is 20:1, a mass concentration of the aqueous solution of sodium bicarbonate is 5%, a molar ratio of the sodium bicarbonate to the nobiletin is 13:1, a mass concentration of the aqueous solution of potassium peroxymonosulfate is 10%, and a molar ratio of the potassium peroxymonosulfate to the nobiletin is 10:1.

(4) adding 1-(3-Dimethylaminopropyl)-3-ethylcarbodiimide hydrochloride (EDCI) and 4-dimethylaminopyridine (DMAP) into a dichloromethane (CH₂Cl₂) solution of the compound C and the compound D to react to thereby obtain a compound E as the synthetic compound of myricetin and nobiletin. A chemical equation IV is as follows:

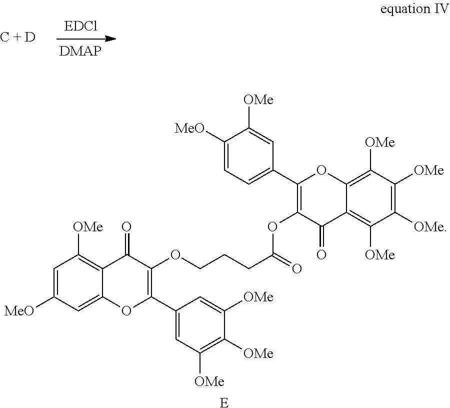

In the step (4), a molar ratio of the compound C to the compound D is 1:1, a volume mass ratio of the dichloromethane solution to the compound C is 100:1, a molar ratio of the 1-(3-Dimethylaminopropyl)-3-ethylcarbodiimide hydrochloride to the compound C is 2:1, and a molar ratio of the 4-dimethylaminopyridine to the compound C is 1:1.

The beneficial effects of the disclosure are as follows.
(1) The two lead compounds with clinical significance are modified to obtain a new synthetic compound of myricetin and nobiletin. Compared with traditional western medicine, the new synthetic compound has significant advantages in treating complications and fewer adverse reactions.
(2) The reduction levels of serum uric acid and creatinine of the synthetic compound of myricetin and nobiletin are significantly higher than that of monomer myricetin and monomer nobiletin, which indicates that the synthetic compound of myricetin and nobiletin has a better uric acid lowering effect than monomeric medicines and has a significant improvement effect on hyperuricemia.

### BRIEF DESCRIPTION OF DRAWINGS

FIG. 1 illustrates a diagram showing effects of different treatments on uric acid (UA) in serums of mice (Mean±standard error of the mean abbreviated as SEM). Note: compared with a normal group, ^{###}P<0.001; compared with a model group, **P<0.01, ***P<0.001.

FIG. 2A illustrates a diagram showing effects of the different treatments on creatinine (CRE) in the serums of the mice (Mean±SEM). Note: compared with a normal group, ^{###}P<0.001; compared with a model group, ***P<0.001.

FIG. 2B illustrates a diagram showing effects of the different treatments on blood urea nitrogen (BUN) in the serums of the mice (Mean±SEM). Note: compared with a normal group, ^{###}P<0.001; compared with a model group, ***P<0.001.

FIG. 3A illustrates a diagram showing effects of the different treatments on kidney index of the mice (Mean±SEM). Note: compared with a normal group, ^{###}P<0.001; compared with a model group, *P<0.05, **P<0.01, ***P<0.001.

FIG. 3B illustrates a diagram showing effects of the different treatments on live index of the mice (Mean±SEM). Note: compared with a normal group, ^{###}P<0.001; compared with a model group, *P<0.05, **P<0.01, ***P<0.001.

FIG. 4 illustrates a diagram showing effects of a synthetic compound on pathological results of renal tissues in hyperuricemia mice through hematoxylin and eosin (HE) staining with a magnification of 200 (×200).

### DETAILED DESCRIPTION OF EMBODIMENTS

### Embodiment 1

The myricitrin (0.8 grams abbreviated as g, 1.72 millimoles abbreviated as mmol) and of the potassium carbonate (1.8 g, 12.9 mmol) are mixed, and added with the acetonitrile solution (40 milliliters abbreviated as mL) to stirred evenly to obtain a first mixed solution, then dimethyl sulfate (1.2 mL, 12.9 mmol) are added into the first mixed solution to react at 85° C. for 24 hours to obtain a reacted product, followed by filtering and desolventizing to obtain the compound A. The compound A is dissolved in ethanol (40 mL), concentrated hydrochloric acid (1.4 mL) are added to react at 70° C. for 12 hours to obtain a first reacted solution, then the ethanol of the first reacted solution is removed by desolvation to obtain a crude product of the compound B, the crude product of the compound B is dissolved in dichloromethane to obtain a dissolved crude product, the dissolved crude product is washed and then dried with anhydrous sodium sulfate, followed by filtering and desolventizing to obtain the compound B for standby.

The potassium carbonate (1.33 g, 9.6 mmol) and the BrCH₂CH₂CH₂CO₂Et (1.25 g, 6.4 mmol) are added into the N,N-dimethylformamide solution (20 mL) of the compound B (1.25 g, 3.2 mmol) to react at 60° C. for 12 hours to obtain a reacted product, then the reacted product is washed with water, followed by adding ethyl acetate to extract and desolventize the washed reacted product to obtain a first intermediate product. The intermediate product is dissolved in tetrahydrofuran (20 mL), an aqueous solution of sodium hydroxide is added into the first intermediate product after dissolving to react at room temperature for 6 hours, followed by desolventizing to obtain an aqueous phase product. A diluted hydrochloric acid is added to adjust a potential of hydrogen (pH) of the aqueous phase product to 4, the adjusted aqueous phase product is extracted by adding dichloromethane to thereby obtain a crude product of the compound C, then the crude product of the compound C is dried with anhydrous sodium sulfate, followed by desolventizing the dried crude product to obtain the compound C.

The 5% aqueous solution of sodium carbonate (79 g) and the 5% aqueous solution of sodium bicarbonate (41 g) are added into the dichloromethane (75 mL) and acetone (56 mL) solution of the nobiletin (0.75 g, 1.86 mmol) into to obtain a second mixed solution, then the 10% aqueous solution of potassium peroxymonosulfate is added into the second mixed solution to react at room temperature for 48 hours to a third reacted product, followed by desolventizing the third reacted product to obtain a crude product of the compound D, the crude product of the compound D is dissolved in dichloromethane, diluted hydrochloric acid is added to wash the dissolved crude product of the compound D, followed by performing liquid separation on the washed crude product of the compound D to obtain an aqueous phase product of the crude product of the compound D, then the aqueous phase product of the crude product of the compound D is extracted by adding dichloromethane to obtain an extracted product, followed by desolventizing the extracted product, and a silica gel column chromatography is performed on the desolventized product to obtain the compound D (315 mg), and a yield of the compound D is 40%.

The 1-(3-Dimethylaminopropyl)-3-ethylcarbodiimide hydrochloride (EDCI, 1.5 mmol) and the 4-dimethylaminopyridine (DMAP, 0.75 mmol) are added into the dichloromethane solution of the compound C (357 mg, 0.75 mmol) and the compound D (315 mg, 0.75 mmol) to react at the room temperature for 12 hours to obtain a fourth reacted product, followed by performing dry sampling and silica gel column chromatography on the fourth reacted product to obtain the compound E (598 mg) as the synthetic compound of myricetin and nobiletin. A yield of the compound E is 91.0%. The compound E is a faint yellow solid, and a melting point of the compound E is 82-84° C. ¹H nuclear magnetic resonance (NMR) (400 MHz, Chloroform-d) δ 7.57 (dd, J=8.6, 2.1 Hz, 1H), 7.48 (d, J=2.1 Hz, 1H), 7.36 (s, 2H), 6.98 (d, J=8.5 Hz, 1H), 6.51 (d, J=2.2 Hz, 1H), 6.37 (d, J=2.3 Hz, 1H), 4.15-4.11 (m, 2H), 4.10 (s, 3H), 4.00-3.91 (m, 30H), 3.02-2.89 (m, 2H), 2.23 (p, J=6.8 Hz, 2H); ¹³C NMR (101 MHz, Chloroform-d) δ 173.43, 170.15, 170.09, 163.53, 160.56, 158.33, 153.14, 152.58, 152.15, 151.16, 150.97, 148.44, 147.96, 146.53, 143.65, 139.99, 137.37, 132.56, 125.49, 121.89, 121.62, 113.97, 110.67, 110.05, 108.95, 105.37, 95.35, 91.98, 70.61, 61.82, 61.46, 61.35, 61.21, 60.49, 55.93, 55.48, 55.34, 30.30, 25.18; HRMS (m/z): calcd for C₄₅H₄₆NaO₁₈ [M+Na]⁺) 897.2576, found 897.2570.

### Embodiment 2

The myricitrin (0.8 g, 1.72 mmol) and of the potassium carbonate (1.43 g, 10.3 mmol) are mixed, and added with the acetonitrile solution (40 mL) to stirred evenly to obtain a first mixed solution, then dimethyl sulfate (1.0 mL, 10.3 mmol) are added into the first mixed solution to react at 85° C. for 24 hours to obtain a reacted product, followed by filtering and desolventizing to obtain the compound A. The compound A is dissolved in ethanol (40 mL), concentrated hydrochloric acid (1.4 mL) are added to react at 70° C. for 12 hours to obtain a first reacted solution, then the ethanol of the first reacted solution is removed by desolvation to obtain a crude product of the compound B, the crude product of the compound B is dissolved in dichloromethane to obtain a dissolved crude product, the dissolved crude product is washed and then dried with anhydrous sodium sulfate, followed by filtering and desolventizing to obtain the compound B for standby.

The potassium carbonate (1.33 g, 9.6 mmol) and the BrCH₂CH₂CH₂CO₂Et (1.25 g, 6.4 mmol) are added into the N,N-dimethylformamide solution (23 mL) of the compound B (1.25 g, 3.2 mmol) to react at 60° C. for 12 hours to obtain a reacted product, then the reacted product is washed with water, followed by adding ethyl acetate to extract and desolventize the washed reacted product to obtain a first intermediate product. The intermediate product is dissolved in tetrahydrofuran (23 mL), an aqueous solution of sodium hydroxide is added into the first intermediate product after dissolving to react at room temperature for 6 hours, followed by desolventizing to obtain an aqueous phase product. A diluted hydrochloric acid is added to adjust a pH of the aqueous phase product to 4, the adjusted aqueous phase product is extracted by adding dichloromethane to thereby obtain a crude product of the compound C, then the crude product of the compound C is dried with anhydrous sodium sulfate, followed by desolventizing the dried crude product to obtain the compound C.

The 5% aqueous solution of sodium carbonate (79 g) and the 5% aqueous solution of sodium bicarbonate (41 g) are added into the dichloromethane (75 mL) and acetone (56 mL) solution of the nobiletin (0.75 g, 1.86 mmol) into to obtain a second mixed solution, then the 10% aqueous solution of potassium peroxymonosulfate is added into the second mixed solution to react at room temperature for 48 hours to a third reacted product, followed by desolventizing the third reacted product to obtain a crude product of the compound D, the crude product of the compound D is dissolved in dichloromethane, diluted hydrochloric acid is added to wash the dissolved crude product of the compound D, followed by performing liquid separation on the washed crude product of the compound D to obtain an aqueous phase product of the crude product of the compound D, then the aqueous phase product of the crude product of the compound D is extracted by adding dichloromethane to obtain an extracted product, followed by desolventizing the extracted product, and a silica gel column chromatography is performed on the desolventized product to obtain the compound D (315 mg), and a yield of the compound D is 40%.

The 1-(3-Dimethylaminopropyl)-3-ethylcarbodiimide hydrochloride (1.5 mmol) and the 4-dimethylaminopyridine (0.75 mmol) are added into the dichloromethane solution of the compound C (357 mg, 0.75 mmol) and the compound D (315 mg, 0.75 mmol) to react at the room temperature for 12 hours to obtain a fourth reacted product, followed by performing dry sampling and silica gel column chromatography on the fourth reacted product to obtain the compound E (598 mg) as the synthetic compound of myricetin and nobiletin. A yield of the compound E is 91.6%.

### Embodiment 3

The myricitrin (0.8 g, 1.72 mmol) and of the potassium carbonate (1.9 g, 13.8 mmol) are mixed, and added with the acetonitrile solution (40 mL) to stirred evenly to obtain a first mixed solution, then dimethyl sulfate (1.3 mL, 13.8 mmol) are added into the first mixed solution to perform a temperature control reaction at 85° C. for 24 hours to obtain a reacted product, followed by filtering and desolventizing to obtain the compound A. The compound A is dissolved in ethanol (40 mL), concentrated hydrochloric acid (1.4 mL) are added to react at 70° C. for 12 hours to obtain a first reacted solution, then the ethanol of the first reacted solution is removed by desolvation to obtain a crude product of the compound B, the crude product of the compound B is dissolved in dichloromethane to obtain a dissolved crude product, the dissolved crude product is washed and then dried with anhydrous sodium sulfate, followed by filtering and desolventizing to obtain the compound B for standby.

The potassium carbonate (1.33 g, 9.6 mmol) and the BrCH₂CH₂CH₂CO₂Et (1.25 g, 6.4 mmol) are added into the N,N-dimethylformamide solution (25 mL) of the compound B (1.25 g, 3.2 mmol) to react at 60° C. for 12 hours to obtain a reacted product, then the reacted product is washed with water, followed by adding ethyl acetate to extract and desolventize the washed reacted product to obtain a first intermediate product. The intermediate product is dissolved in tetrahydrofuran (25 mL), an aqueous solution of sodium hydroxide is added into the first intermediate product after dissolving to react at room temperature for 6 hours, followed by desolventizing to obtain an aqueous phase product. A diluted hydrochloric acid is added to adjust a pH of the aqueous phase product to 4, the adjusted aqueous phase product is extracted by adding dichloromethane to thereby obtain a crude product of the compound C, then the crude product of the compound C is dried with anhydrous sodium sulfate, followed by desolventizing the dried crude product to obtain the compound C.

The 5% aqueous solution of sodium carbonate (79 g) and the 5% aqueous solution of sodium bicarbonate (41 g) are added into the dichloromethane (75 mL) and acetone (56 mL) solution of the nobiletin (0.75 g, 1.86 mmol) into to obtain a second mixed solution, then the 10% aqueous solution of potassium peroxymonosulfate is added into the second mixed solution to react at room temperature for 48 hours to a third reacted product, followed by desolventizing the third reacted product to obtain a crude product of the compound D, the crude product of the compound D is dissolved in dichloromethane, diluted hydrochloric acid is added to wash the dissolved crude product of the compound D, followed by performing liquid separation on the washed crude product of the compound D to obtain an aqueous phase product of the crude product of the compound D, then the aqueous phase product of the crude product of the compound D is extracted by adding dichloromethane to obtain an extracted product, followed by desolventizing the extracted product, and a silica gel column chromatography is performed on the desolventized product to obtain the compound D (315 mg), and a yield of the compound D is 40%.

The 1-(3-Dimethylaminopropyl)-3-ethylcarbodiimide hydrochloride (1.5 mmol) and the 4-dimethylaminopyridine (0.75 mmol) are added into the dichloromethane solution of the compound C (357 mg, 0.75 mmol) and the compound D (315 mg, 0.75 mmol) to react at the room temperature for 12 hours to obtain a fourth reacted product, followed by performing dry sampling and silica gel column chromatography on the fourth reacted product to obtain the compound E (598 mg) as the synthetic compound of myricetin and nobiletin. A yield of the compound E is 90.8%.

### Experimental Embodiment 1

### Effects of Synthetic Compound of Myricetin and Nobiletin on Animal Models With Hyperuricemia

### 1.1 Materials

### 1.1.1 Experimental Animals and Feeds

Specific pathogen free (SPF) grade KunMing (KM) mice, male, aged 5-6 weeks, weighing 22-25 g, are provided by Medical Experimental Animal Center of Guangdong Province with an animal qualification certificate number: SCXK (Yue) 2022-0002. The mice are raised in plastic cages, with free diet and drinking water. The indoor temperature of the plastic cages is (22±2)° C., and a relative humidity is maintained at 55%±5%. The lights are turned on and turned off for 12 hours, respectively. After quarantine and adaptation for one week, relevant experiments are conducted. The experimental plan is reviewed and approved by the Animal Welfare Ethics Committee of Zhongke Industry Holdings (Shenzhen) Co., Ltd. (application number: 20220082), and the operation and process of animal experiments follow the “Guidelines for the use of experimental animals in teaching” of the Chinese Society of Experimental Animals.

Customized dietary feeds: 15% of yeast extract feeds, in compliance with regulations of GB13078 and GB14924.2, are provided by Jiangsu Synergy Pharmaceutical Biotechnology Co., Ltd. Production license: Su Si Zheng (2019) 01008.

### 1.1.2 Medicine and Reagents

The synthetic compound of myricetin and nobiletin is synthesized and self-made in the laboratory. Isoflurane (RWD Life Science Co., LTD); uric acid detection assay kit (Nanjing Jiancheng Biotechnology Research Institute, batch number: 20221013), creatinine (Cr) determination test kit (Nanjing Jiancheng Biotechnology Research Institute, batch number: 20221015), urea nitrogen (BUN) content assay kit (Beijing Solaibao Technology Co., Ltd., batch number: 20221014).

### 1.2 Experimental Methods

### 1.2.1 Animal Grouping and Administration

After 2 weeks of adaptive feeding in the plastic cages, the SPF grade KM mice are randomly divided into five groups: normal group (Control), model group (Model), synthetic compound group (PJW), myricetin group (Myricetin), and nobiletin group (Nobiletin). The dosage of each individual test medicine group is 30 milligrams per kilogram (mg/kg), and the medicines are dissolved in a 0.5% carboxymethyl cellulose sodium solution with a gavage volume of 10 milliliters per kilogram (mL/kg). Except for the normal group fed with basic feed, the other groups are fed with 15% of the yeast extract feeds for modeling. The normal group and the model group are intragastrically administered an equal amount of physiological saline daily, and the other groups are intragastrically administered with the corresponding medicine daily for 9 weeks.

### 1.2.2 Sample Collection and Serum Biochemical Index Detection

At the end of the experiment, that is, at the end of the 9^{th} week of administration, the KM mice are fasted for 12 hours, then the KM mice are anesthetized with the isoflurane to take off the eyeballs from the KM mice for blood collection, followed by placing the collected blood to stand in the room temperature for 1 hour. After the standing, the blood is centrifuged at 3500 revolutions per minute (rpm/min) for 15 minutes to obtain centrifuged solutions, respectively, then serums of the centrifuged solution are separated to detect the levels of the uric acid, the creatinine, the urea nitrogen. The KM mice are sacrificed by dislocation of cervical vertebra after collecting blood, livers and kidneys are dissected from the KM mice and weighed to calculate indexes of the livers and the kidneys, an organ index of the formula is as follows: organ index=organ weight (g)/body mass (g)×100%. Then the livers and the kidneys are quickly packed, one portion of the packed sample is fixed with 4% paraformaldehyde solution for HE staining, and the other portion of the packed sample is stored at −80° C. in a refrigerator for later use.

### 1.2.3 Pathological Observation of Renal Tissue

The renal tissues fixed with 4% paraformaldehyde from the kidneys are taken to be dehydrated with ethanol, embedded in paraffin, and sliced. After staining with hematoxylin-eosin, the pathological changes of renal tissues are observed under an upright optical microscope.

### 1.2.4 Statistical Methods

All data are statistically analyzed using a GraphPad Prism 7.00 software, and the data are presented as a mean value±a standard error (standard deviation±standard error of the mean abbreviated as SD±SEM). Multiple group comparisons are conducted using a one-way Analysis of Variance (ANOVA), and pairwise comparisons between groups are conducted using a Dunnett's test. P<0.05 indicates a statistically significant difference.

### 1.3 Results

### 1.3.1 Effect of Synthetic Compound on Serum Uric Acid (UA) in Mice With Hyperuricemia

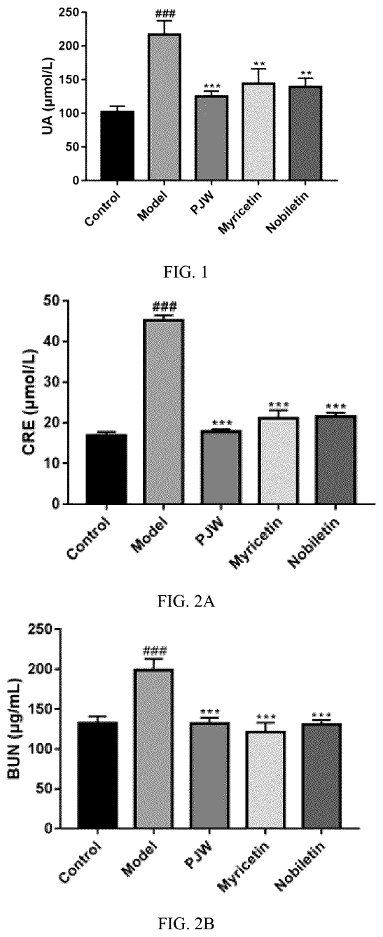

Compared with the normal group, the UA level of the model group mice is significantly increased (P<0.001), indicating the success of the model. Compared with the model group, the serum UA levels of mice in the synthetic compound group, the myricetin group, and the nobiletin group are significantly reduced (P<0.001) (P<0.001, P<0.01), and the effect of the synthetic compound group is better than that of each monomer group. As shown in FIG. 1.

### 1.3.2 Effect of Synthetic Compound on Serum Creatinine and Urea Nitrogen in Mice With Hyperuricemia

Compared with the normal group, the serum creatinine level and the urea nitrogen level of the model group mice are significantly increased (P<0.001), indicating the success of the model. Compared with the model group, the serum creatinine level and the urea nitrogen level of mice in the synthetic compound group, the myricetin group, and the nobiletin group are significantly reduced (P<0.001), and the effect of the synthetic compound on creatinine is better than that of monomer medicines. As shown in FIGS. 2A-2B.

### 1.3.3 Effect of Synthetic Compound on Organ Index in Mice With Hyperuricemia

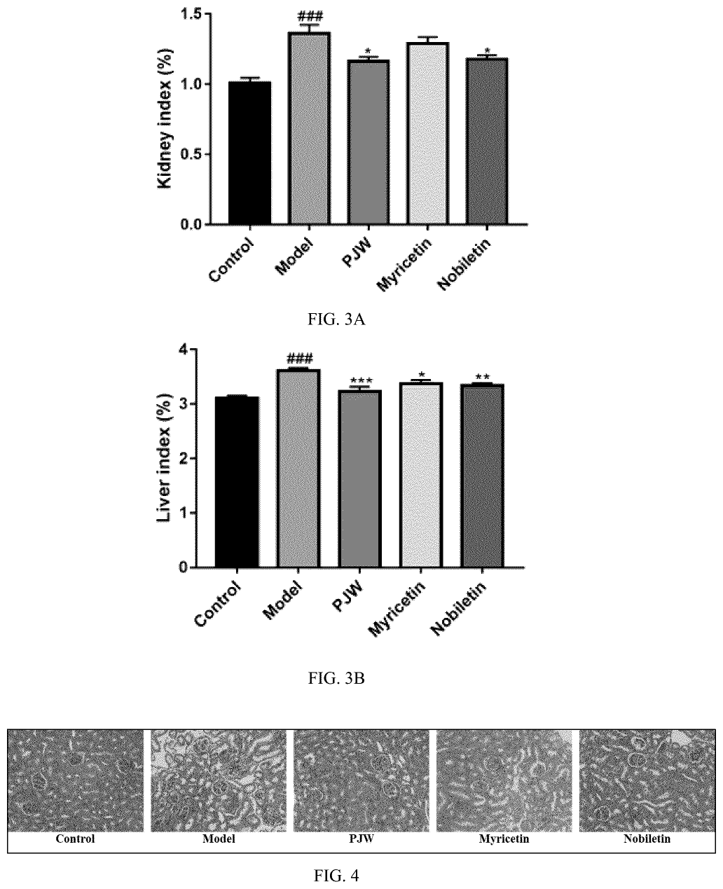

Compared with the normal group, the kidney indexes and liver indexes of the model group mice are significantly increased, indicating that the yeast extract induced hyperuricemia nephropathy model in mice is successfully modeled. Compared with the model group, the kidney indexes of mice of the synthetic compound group and the nobiletin group are significantly reduced (P<0.05, P<0.001), and the liver indexes of mice of the synthetic compound group, the myricetin group, and the nobiletin group are significantly reduced (P<0.001, P<0.05, P<0.01), and the effect of the synthetic compound group is better than that of each monomer group. As shown in FIGS. 3A-3B.

### 1.3.4 Effect of Synthetic Compound on Renal Morphological Changes in Hyperuricemia Model Mice

The HE staining results showed that in the normal group of mice, the distribution of glomeruli in the renal cortex is uniform, the arrangement of cells is neat, there is no proliferation of the mesangial matrix, the epithelial cells of renal tubules are round and full, and brush-like edges are arranged regularly, and the cortical and medullary parts are normal, without occlusion, expansion, atrophy, or necrosis. In the mice of the model group, the renal tubular epithelial cells are edematous and degenerated, characterized by increased cell volume, loose cytoplasm with light staining, and unclear lumen margins. The synthetic compound, the myricetin, and the nobiletin can improve renal injury in mice with hyperuricemia to varying degrees, mainly manifested by no significant degeneration of renal tubular epithelial cells and no significant dilation of the lumen. It can be seen that the improvement effect of the synthetic compound on renal pathological morphology is better than that of monomer medicines. As shown in FIG. 4.

What is claimed is:

1. A synthetic compound of myricetin (C₁₅H₁₀O₈) and nobiletin (C₂₇H₃₂O₁₄), wherein a structural general formula of the synthetic compound of myricetin and nobiletin is shown in a formula E expressed as follows: [symbol]
formula E.

2. A preparation method for the synthetic compound of myricetin and nobiletin as claimed in claim 1, comprising the following steps:
(1) mixing myricitrin (C₂₁H₂₀O₁₂) and potassium carbonate (K₂CO₃) in an acetonitrile (C₂H₃N) solution to obtain a first mixed solution, then adding dimethyl sulfate (Me₂SO₄) into the first mixed solution to perform a temperature control reaction to obtain a compound A, and dissolving the compound A in ethanol, followed by adding a concentrated hydrochloric acid (conc. HCl) to react to obtain a compound B; wherein a chemical equation I is as follows: [symbol]
equation I;
(2) adding the potassium carbonate and BrCH₂CH₂CH₂CO₂Et into a N,N-dimethylformamide (C₃H₇NO) solution of the compound B to perform a temperature control reaction to obtain a compound C; wherein a chemical equation II is as follows: 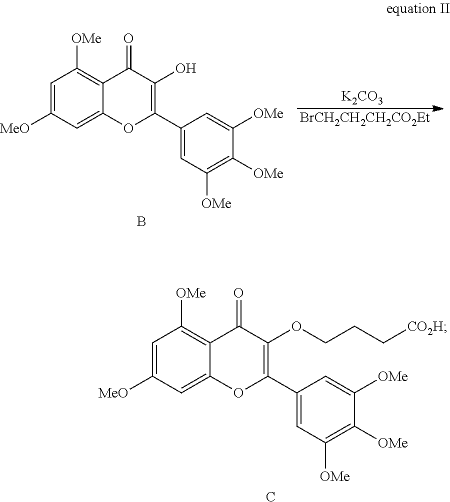
(3) adding an aqueous solution of sodium carbonate (Na₂CO₃) and an aqueous solution of sodium bicarbonate (NaHCO₃) into a dichloromethane (CH₂Cl₂) and acetone (C₃H₆O) solution of the nobiletin to obtain a second mixed solution, followed by adding an aqueous solution of potassium peroxymonosulfate (HKO₆S) into the second mixed solution to obtain a compound D; wherein a chemical equation III is as follows: [symbol]
equation III;
(4) adding 1-(3-Dimethylaminopropyl)-3-ethylcarbodiimide hydrochloride (EDCI) and 4-dimethylaminopyridine (DMAP) into a dichloromethane (CH₂Cl₂) solution of the compound C and the compound D to react to thereby obtain a compound E as the synthetic compound of myricetin and nobiletin; wherein a chemical equation IV is as follows:

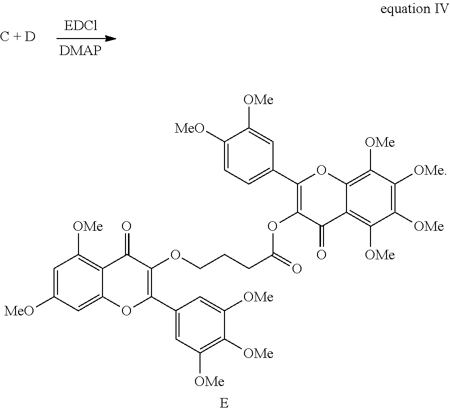

3. The preparation method as claimed in claim 2, wherein in the step (1), a molar ratio of the myricitrin to the potassium carbonate is in a range of 1:6-8, a volume mass ratio of the acetonitrile solution to the myricitrin is 50:1, a molar ratio of the dimethyl sulfate to the potassium carbonate is 1:1; a volume ratio of the acetonitrile solution to the ethanol used to dissolve the compound A is 1:1, a mass concentration of the concentrated hydrochloric acid is 37% in terms of hydrogen chloride (HCl), and a molar ratio of the concentrated hydrochloric acid to the myricetin is 10:1.

4. The preparation method as claimed in claim 2, wherein in the step (2), a volume mass ratio of the compound B to the N,N-dimethylformamide solution is in a range of 1:15-20, a molar ratio of the potassium carbonate to the compound B is 3:1, a molar ratio of the BrCH₂CH₂CH₂CO₂Et to the compound B is 2:1, and a temperature of the temperature control reaction is 60° C.

5. The preparation method as claimed in claim 2, wherein in the step (3), a volume mass ratio of the dichloromethane and acetone solution to the nobiletin is 175:1, a volume ratio of the dichloromethane to the acetone is 4:3, a mass concentration of the aqueous solution of sodium carbonate is 5%, a molar ratio of the sodium carbonate to the nobiletin is 20:1, a mass concentration of the aqueous solution of sodium bicarbonate is 5%, a molar ratio of the sodium bicarbonate to the nobiletin is 13:1, a mass concentration of the aqueous solution of potassium peroxymonosulfate is 10%, and a molar ratio of the potassium peroxymonosulfate to the nobiletin is 10:1.

6. The preparation method as claimed in claim 2, wherein in the step (4), a molar ratio of the compound C to the compound D is 1:1, a volume mass ratio of the dichloromethane solution to the compound C is 100:1, a molar ratio of the 1-(3-Dimethylaminopropyl)-3-ethylcarbodiimide hydrochloride to the compound C is 2:1, and a molar ratio of the 4-dimethylaminopyridine to the compound C is 1:1.

7. The preparation method as claimed in claim 2, wherein
the step (1) specifically comprises:
mixing the myricitrin and the potassium carbonate in the acetonitrile solution to obtain the first mixed solution, then adding the dimethyl sulfate into the first mixed solution to perform the temperature control reaction at 85° C. for 24 hours to obtain a reacted product, followed by filtering and desolventizing the reacted product to obtain the compound A; dissolving the compound A in the ethanol, adding the concentrated hydrochloric acid to react at 70° C. for 12 hours to obtain a first reacted solution, then removing the ethanol of the first reacted solution by desolvation to obtain a crude product of the compound B, dissolving the crude product of the compound B in dichloromethane to obtain a dissolved crude product, washing the dissolved crude product and drying the washed crude product with anhydrous sodium sulfate, followed by filtering and desolventizing to obtain the compound B for standby;
the step (2) specifically comprises:
adding the potassium carbonate and the BrCH₂CH₂CH₂CO₂Et into the N,N-dimethylformamide solution of the compound B to perform the temperature control reaction at 60° C. for 12 hours to obtain a reacted product, then washing the reacted product with water, followed by adding ethyl acetate to extract and desolventize the washed reacted product to obtain a first intermediate product; dissolving the intermediate product in tetrahydrofuran (C₄H₈O), adding an aqueous solution of sodium hydroxide (NaOH) into the first intermediate product after dissolving to react at room temperature for 6 hours, followed by desolventizing to obtain an aqueous phase product; adding a diluted hydrochloric acid to adjust a potential of hydrogen (pH) of the aqueous phase product to 4, extracting the adjusted aqueous phase product by adding dichloromethane to thereby obtain a crude product of the compound C, then drying the crude product of the compound C with anhydrous sodium sulfate, followed by desolventizing the dried crude product to obtain the compound C;
the step (3) specifically comprises:
adding the aqueous solution of sodium carbonate and the aqueous solution of sodium bicarbonate into the dichloromethane and acetone solution of the nobiletin into to obtain the second mixed solution, then adding the aqueous solution of potassium peroxymonosulfate into the second mixed solution to react at room temperature for 48 hours to a third reacted product, followed by desolventizing the third reacted product to obtain a crude product of the compound D, dissolving the crude product of the compound D in dichloromethane, adding diluted hydrochloric acid to wash the dissolved crude product of the compound D, followed by performing liquid separation on the washed crude product of the compound D to obtain an aqueous phase product of the crude product of the compound D, then extracting the aqueous phase product of the crude product of the compound D by adding dichloromethane to obtain an extractd product, followed by desolventizing the extracted product, and performing a silica gel column chromatography on the desolventized product to obtain the compound D;
the step (4) specifically comprises:
adding the 1-(3-Dimethylaminopropyl)-3-ethylcarbodiimide hydrochloride and the 4-dimethylaminopyridine into the dichloromethane solution of the compound C and the compound D to react at the room temperature for 12 hours to obtain a fourth reacted product, followed by performing dry sampling and silica gel column chromatography on the fourth reacted product to obtain the compound E as the synthetic compound of myricetin and nobiletin.

8. The preparation method as claimed in claim 7, wherein in the step (2), a volume ratio of the tetrahydrofuran to the N, N-dimethylformamide is 1:1, a mass concentration of the aqueous solution of sodium hydroxide is in a range of 5%-40%, a molar ratio of the sodium hydroxide to the compound B is 2:1, and a molar concentration of the dilute hydrochloric acid is 4 moles per liter (mol/L).

9. An application method of the synthetic compound of the myricetin and the nobiletin as claimed in claim 1, comprising:
applying a medicine prepared by using the synthetic compound of the myricetin and the nobiletin to lower uric acid to treat a patient with hyperuricemia.
